(19) United States
(12) Patent Application Publication
(10) Pub. No.: US 2017/0172561 A1
(43) Pub. Date: Jun. 22, 2017

(54) HYBRID SUTURE ANCHOR

(71) Applicant: Biomet Sports Medicine, LLC, Warsaw, IN (US)

(72) Inventor: Gregory J. Denham, Warsaw, IN (US)

(21) Appl. No.: 15/355,714

(22) Filed: Nov. 18, 2016

Related U.S. Application Data
(60) Provisional application No. 62/257,423, filed on Nov. 19, 2015.

(51) Int. Cl. A61B 17/04

(52) U.S. Cl. CPC A61B 17/0401; A61B 2017/0409; A61B 2017/0458; A61B 2017/042; A61B 2017/044; A61B 2017/00004

(57) ABSTRACT

An anchor having a nose portion and a tail portion insertable into a bore hole of in a bone, where the tail portion includes radial fixation features for engaging the inner surface of the bore hole when the tail portion is inserted into the bore hole. A suture can be attached to the nose portion through a through hole in the tail portion. The suture can be tensioned to radially expand the nose portion into engagement with the inner surface of the bore hole. The rigid tail portion provides a backstop limiting axial movement of the nose portion when the suture is tensioned to prevent the nose portion from being inadvertently withdrawn from the bore hole. The rigid tail portion can also increase the amount of tension applied to the suture and correspondingly the radial expansion of the nose portion.

### CLAIM OF PRIORITY

This patent application claims the benefit of priority, under 35 U.S.C. Section 119(e), to Gregory J. Denham U.S. Patent Application Ser. No. 62/257,423, entitled “HYBRID SUTURE ANCHOR,” filed on Nov. 19, 2015 (Attorney Docket No. 4394.E48PRV), which is hereby incorporated by reference herein in its entirety.

### TECHNICAL FIELD

This document pertains generally, but not by way of limitation, to suture anchors for securing sutures to a bone or other rigid structure.

### BACKGROUND

Soft tissue, such as cartridge, ligaments, or muscles, can become tom or detach from the adjoining bones from trauma or disease. The torn soft tissue is re-secured to the bone with a suture that is anchored to the bone such that the torn soft tissue rejoins to the bone or remaining tissue. The suture is commonly anchored to the bone by boring a hole into the bone and fixing an anchor for a suture within the bore hole. The anchors can have threads or expanding features that engage the inner surface of the bore hole to prevent reversal of the suture anchor. The expanding features are commonly deployed by attaching a suture to anchor and pre-tensioning the suture to reverse the anchor such that the expanding features expand to set the anchor. Following fixation of the anchor, the suture can be attached to the soft tissue and tensioned to draw the soft tissue into place. The pre-tensioning of the suture to deploy the expanding features or the tensioning of the suture to draw soft tissue into place can cause the suture to disconnect from the anchor or inadvertently pull the anchor from the bore before the expanding features fully deploy.

### Overview

The present inventor has recognized, among other things, that a problem to be solved can include maintaining an anchor within a bore hole formed in a bone when an attached suture is tensioned to set the anchor within the bore hole. In an example, the present subject matter can provide a solution to this problem, such as by providing an anchor having an expandable nose portion and a tail portion defining an internal channel, wherein a suture can be coupled to the expandable nose portion through the internal channel of the tail portion. The tail portion can include fixation features extending radially outward to engage an inner surface of the bore hole when the tail portion is inserted into the bore hole. Upon setting of the tail portion, the suture can be tensioned to cause the nose portion to expand radially against the inner surface of the bore hole. The setting of the tail portion with the fixation features can permit the tail portion to act as a backstop preventing the nose portion from being inadvertently drawn from the bore hole when the suture is tensioned to expand the nose portion. The tail portion can also increase the amount of tension that can be applied to the suture improving the radial expansion of the nose portion, which improves the engagement of the nose portion to the inner surface of the bore hole.

In an example, an anchor can include a nose portion and a tail portion. The nose portion can include a flexible element configured to receive a suture. The tail portion can include a body defining at least one axial through hole and include at least one radial fixation feature on an exterior surface of the body. The suture can extend through the axial internal channel in the tail portion such that tensioning the suture moves the nose portion axially toward the tail portion. The suture can be tensioned to move the nose portion axially against the tail portion to radially expand the nose portion. In operation, the anchor can be inserted into a bore hole with the nose portion being inserted first followed by the tail portion. The radial fixation features can be sized such that the radial fixation features engage an inner surface of the bore hole. The tail portion can operate as a backstop preventing the nose portion from being withdrawn from the bore hole when the suture is tensioned. The nose portion can be pulled against the tail portion to assist in the radial expansion of the flexible element as the suture is tensioned.

In an example, a suture anchor for anchoring a suture to a bore hole of a bone, can include a nose portion and a tail portion. The nose portion can include at least one flexible element defining a longitudinal bore. The tail portion can include a body having an outer surface and defining an internal channel. The body can be sized to engage the outer surface to an inner surface of the bore hole to fix the tail portion within the bore hole. The suture can be threaded through the internal channel of the tail portion and the longitudinal bore of the flexible element such that the suture can be tensioned through the tail portion to brace each flexible element against the tail portion and radially expand the flexible element against the inner surface of the bore hole to fix the nose portion within the bore hole.

In an example, a method of anchoring a suture to a bone can include boring a bore hole into the bone. The method can also include inserting a nose portion into the bore hole. A suture can be threaded through an internal channel of a tail portion and looped through a longitudinal bore defined by at least one flexible element of the nose portion. The method can also include inserting the tail portion such that an outer surface defined by the tail portion engages an inner surface of the bore hole to fix the tail portion within the bore hole. The method can also include tensioning the suture to draw each flexible element against the tail portion to radially expand the flexible element to engage the inner surface of the bore hole to fix the nose portion within the bore hole.

This overview is intended to provide an overview of subject matter of the present patent application. It is not intended to provide an exclusive or exhaustive explanation of the present subject matter. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1 is a cross-sectional side view of an anchor system having a nose portion and a tail portion positioned within a bore hole in a bone prior to deployment of the nose portion in accordance with an example of the present disclosure.

FIG. 2 is a cross-sectional side view of the anchor system depicted in FIG. 1 with the nose portion deployed within the bore hole to engage an inner surface of the bore hole in accordance with an example of the present disclosure.

FIG. 3 is a schematic diagram illustrating a method of fixing an anchor system to a bone structure according to an example of the present disclosure.

FIG. 4A is a schematic side view illustrating preparation of a bore hole in a bone for receiving an anchor system according to an example of the present disclosure.

FIG. 4B is a schematic side view illustrating positioning an anchor system for insertion within a bore hole according to an example of the present disclosure.

FIG. 4C is a schematic side view illustrating insertion of the anchor system depicted in FIG. 4B within the bore hole such that a tail portion of the anchor system is engaged to an inner surface of the bore hole according to an example of the present disclosure.

FIG. 4D is a schematic side view illustrating deployment of a nose portion of the anchor system depicted in FIG. 4B such that the nose portion radially expands to engage the inner surface of the bore hole according to an example of the present disclosure.

FIG. 5 is a schematic view of a nose portion of an anchor system according to an example of the present disclosure.

FIG. 6 is a schematic view of a nose portion of an anchor system according to an example of the present disclosure.

FIG. 7A is a side view of a tail portion of an anchor system according to an example of the present disclosure.

FIG. 7B is a cross-sectional side view of the tail portion of the anchor system depicted in FIG. 7A along axis Y according to an example of the present disclosure.

FIG. 8A is a side view of a tail portion of an anchor system according to an example of the present disclosure.

FIG. 8B is a cross-sectional side view of the tail portion of the anchor system depicted in FIG. 8A along axis Z according to an example of the present disclosure.

### DETAILED DESCRIPTION

The devices and methods of the present disclosure can be used for various orthopedic applications including soft tissue repair; attaching or re-attaching soft tissue to bone; attaching sutures or other anchors to bone; any other repair of fibrous tissue, such as muscle, ligament or tendon in an arthroscopic or other open procedure. Additional examples can include rotator cuff reconstruction, acromioclavicular (AC) reconstruction, anterior cruciate ligament reconstruction (ACL), and more generally, the fastening of tendons, grafts, or strands to fibrous tissue and bone. Additionally, the devices and methods of the present disclosure can be used for repairing tissue in cardiological, laparoscopic, urological, plastic or other procedures.

As depicted in FIGS, 1-2, a suture anchor system 20, according to an example of the present disclosure, can include a nose portion 22 and a tail portion 24. The anchor system 20 can be engaged to an inner surface 14 of a bore hole 12 formed in a bone 10 to provide an anchor point for a suture 26, where the suture 26 can be attached to soft tissue, bone, or other anchor systems 20. The anchor system 20 can be inserted into the bore hole 12 with the nose portion 22 being inserted first and followed by the tail portion 24. In an example, the tail portion 24 can be first positioned and fixed within the bore hole 12 with the nose portion 22 and attached suture 26 being inserted through an internal channel 40 of the tail portion 26. In this configuration, the tail portion 24 of a plurality of anchor systems 20 can be present prior to the attachment of the sutures 26 to the respective tail portions 24. The tail portion 24 can be configured to engage the inner surface 14 of the bore hole 12 to fix the tail portion 24 within the bore hole 12. A suture 26 can be threaded through the tail portion 24 and operably connected to the nose portion 22. The suture 26 can be tensioned to draw the nose portion 22 axially within the bore hole 12 against the tail portion 24 and radially expand the nose portion 22 against the inner surface 14 of the bore hole 12. The tail portion 24 can act as a back stop preventing the nose portion 22 from being withdrawn from the bore hole 12 by the tensioning of the suture 26.

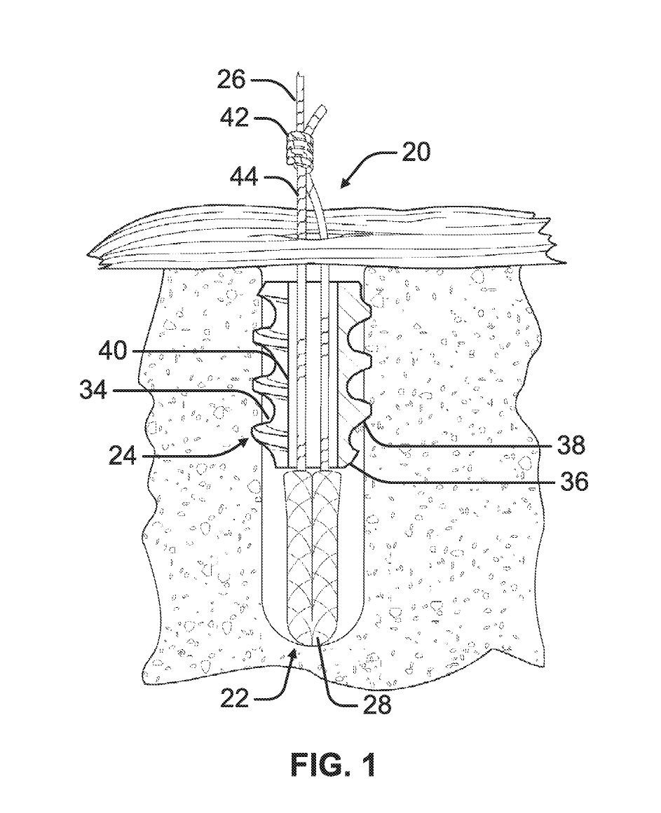

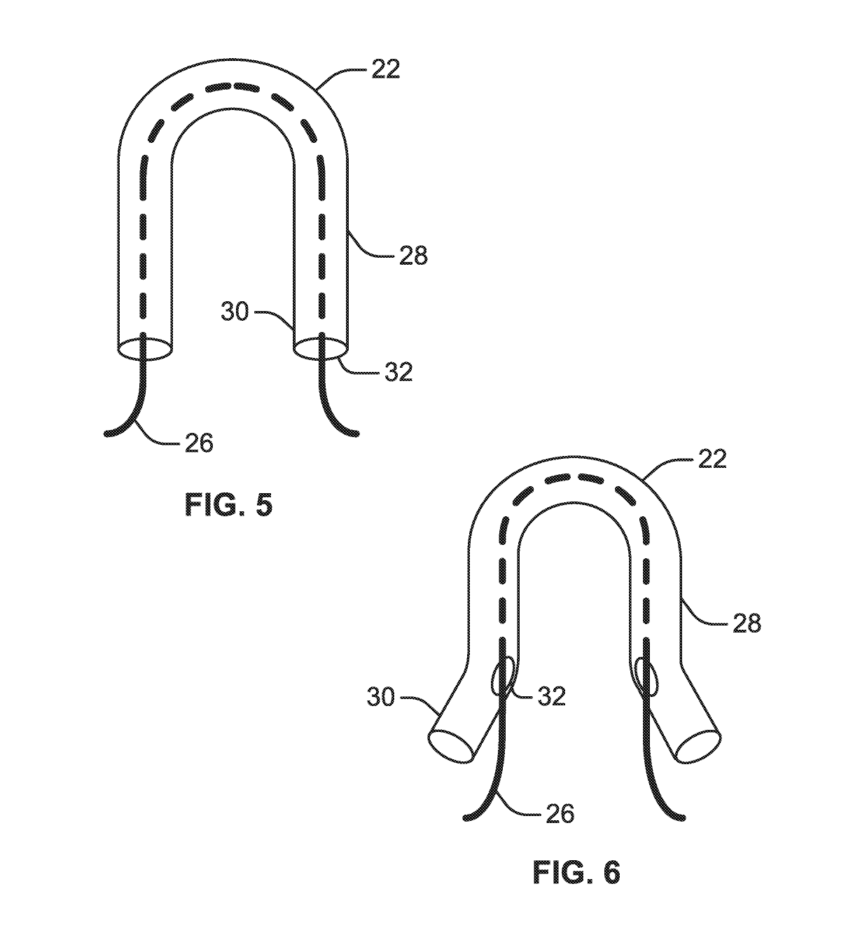

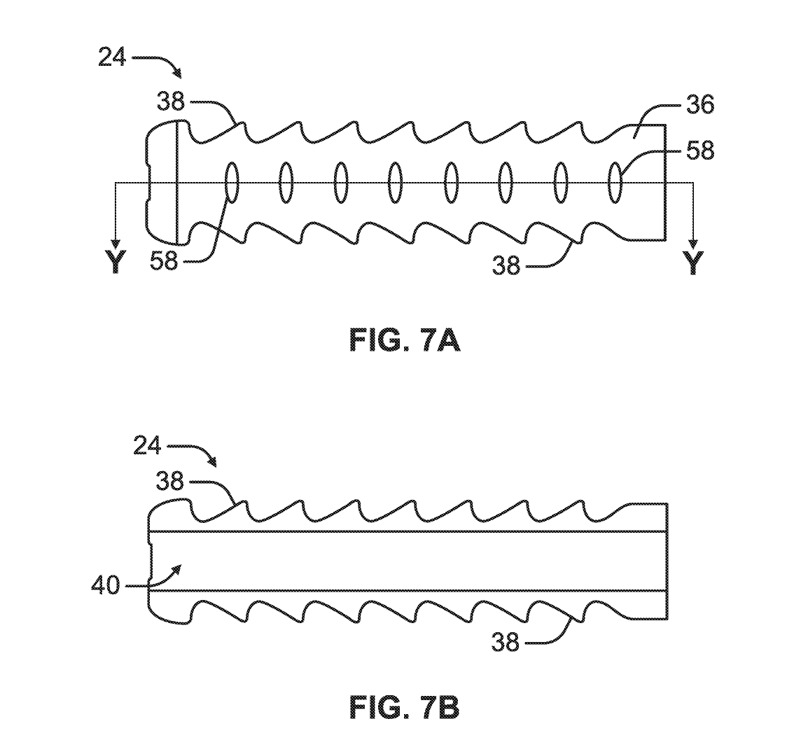

As depicted in FIGS. 1-2 and 5-6, in an example, the nose portion 22 can include at least one flexible element 28 having a longitudinal bore extending between two ends 30. The suture 26 can be threaded through the longitudinal bore to operably connect the suture 26 to the nose portion 22. Each flexible element 22 can include at least two aperture 32 permitting threading of the suture 26 into the longitudinal bore and out of the longitudinal bore. The two apertures 32 can be positioned at the ends 30 of the flexible ends 28, as illustrated in FIG. 5, or positioned inwardly from the two ends 30 such that a portion of the flexible element 28 extend outward from the apertures 32, as illustrated in FIG. 6. In at least one example, one aperture 32 can be positioned immediately adjacent one end 30 and at least one aperture 32 is positioned inward from the other end 30.

In an example, the flexible element 28 can comprise resorbable or non-resorbable materials, including a braided suture, sponges and sponge-like, perforated materials, woven/braided from biocompatible materials or fibers, such as, for example, polymer, polyester, polyethylene, cotton, silk, or other natural or synthetic materials. The flexible element 28 can be made of suture material braided from thin filaments into a form that does not include a core filament. The flexible element 28 can have a generally flaccid shape that can be manipulated in different configurations like a piece of string or shoelace, for example. Accordingly, the flexible element 28 can be bent, folded or otherwise manipulated or deformed into various configurations, such as a bent or U-shape configuration; or a substantially straight configuration; or a deformed or bunched-up, puckered configuration, such as a ball-like or bell-like configuration.

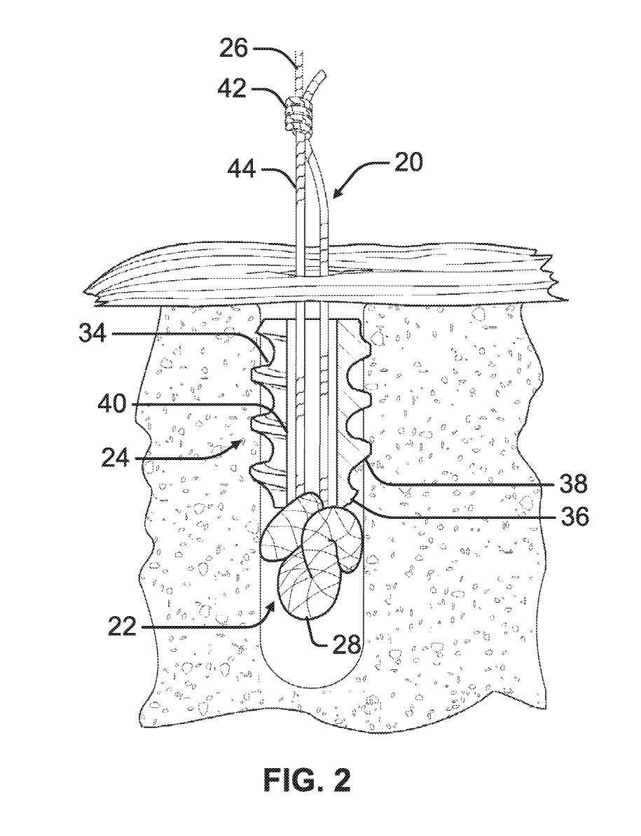

As depicted in FIGS. 1-2, the tail portion 24 can include a body 34 having an outer surface 36 and an inner surface 38 defining an internal channel 40. The body 34 can be sized such that the outer surface 36 approximates the diameter of the bore hole 12 to frictionally engage the inner surface 14 of the bore hole 12. The frictional engagement of the outer surface 36 of the body 34 to the inner surface 14 of the bore hole 12 prevents axial movement of the body 34 within the bore hole 12.

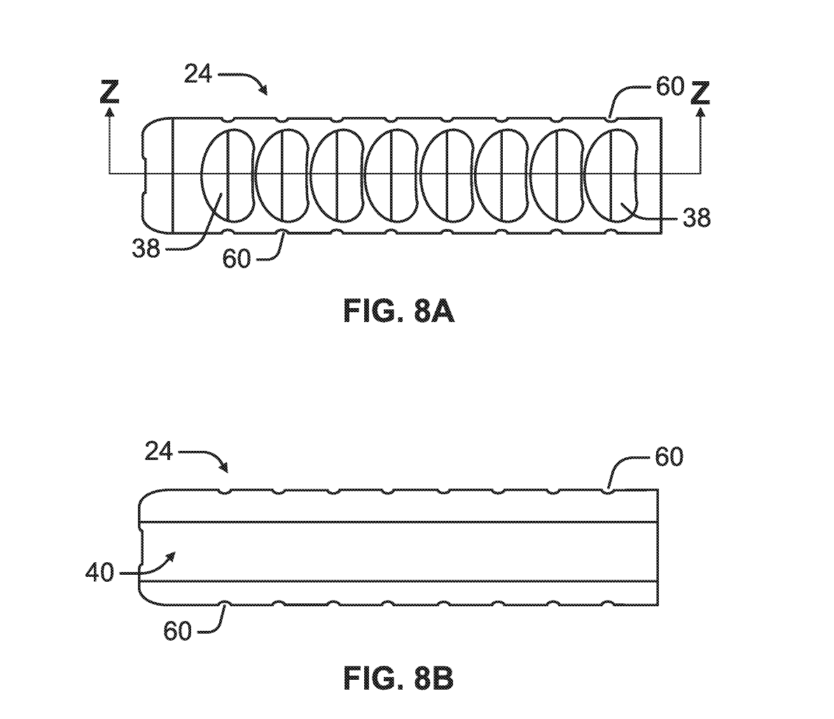

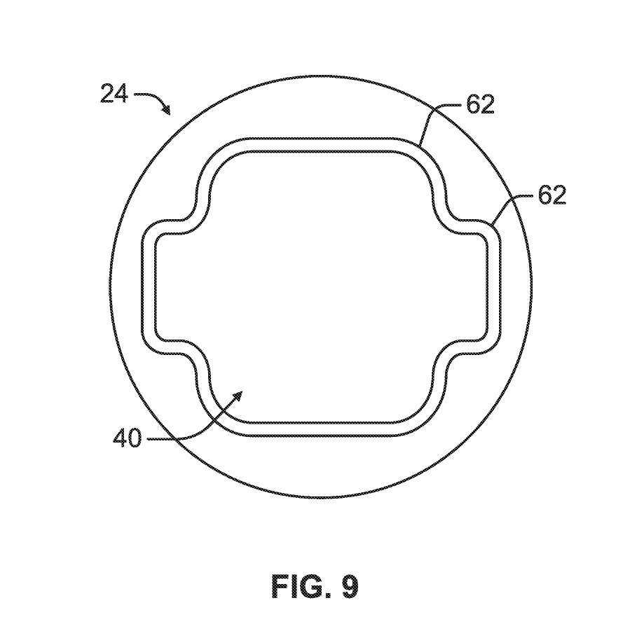

As depicted in FIGS. 1-2, 7A-B, and 8A-B, in an example, the body 34 can include at least one fixation feature 38 extending radially outward from the outer surface 36. The fixation feature 38 can engage the inner surface 14 of the bore hole 12 during or after insertion of the body 34 into the bore hole 12 in a first direction to prevent movement of the body 34 in a second, opposing direction to withdraw the body 34 from the bore hole 12. The at least one fixation feature can comprise barbed rings (as depicted in FIGS. 7A-B); radial projections (as depicted in FIGS. 8A-B); radially deployable features; or other fixation features permitting axial insertion of the body 34 and engagable to the inner surface 14 of the bore hole 12.

As depicted in FIGS. 7A-B, in an example, the body 34 can include at least one opening 58 for texturing the outer surface 36 of the body 34 and/or permitting bone in-growth into the body 34. The texturing provided by the opening 58 can improve engagement of the outer surface 36 of the body 34 to the inner surface 14 of the bore hole 12. As depicted in FIGS. 8A-8B, in an example, the body 34 can include at least one recess 60 for texturing the outer surface 36 of the body 34 and facilitating bone in-growth into the body 34.

As depicted in FIGS. 1-2, in an example, the suture 26 can be threaded through the internal channel 40 of the tail portion 24 and through each flexible element 28 before returning through the internal channel 40 of the tail portion 24. The suture 26 can comprise materials similar to the flexible element 28, such as braided filaments or fibers of biocompatible material, including natural and synthetic fibers, such as cotton, silk, polymer, polyester, polyethylene, thin wire, suture and other materials. The suture 26 can also be in the form of a flat tubular suture or a braded suture with or without a core. As depicted in FIG. 9, in an example, the internal channel 40 can have at least one guide channel 62 for guiding the suture 26 within the tail portion 24 during tensioning of the suture 26. The internal channel 40 can have different sized internal guide channel 62 as illustrated in FIG. 9.

As depicted in FIGS. 1-2, in an example, the ends of the suture 26 can be connected with a knot 42, such as a slipknot, forming a strand loop 44 passing through the internal channel 40 of the tail portion and the longitudinal bore of each flexible element 28. The knot 42 can be threaded through flexible element and tied during the surgical procedure or pre-tied.

The suture anchor system 20 can be used to fasten any type of ligaments, grafts or sutures, and can be used, for example, for rotator cuff repair for the shoulder, for acromioclavicular (AC) joint reconstruction, for tibial graft fixation, for ACL reconstruction, and generally for fastening tendons or grafts and sutures to tissue, including soft tissue and bone. In many of such shoulder repair procedures, a tendon is secured to the bone with many suture anchors requiring repeated knot-typing. Such knot-tying is cumbersome and time consuming during an arthroscopic procedure, as it is generally performed through an insertion cannula 46 that is used to deliver the suture anchor system 20.

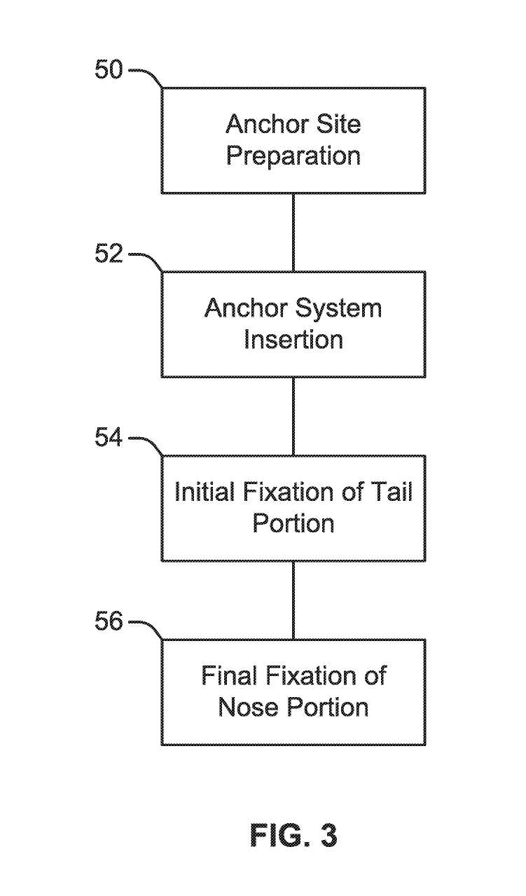

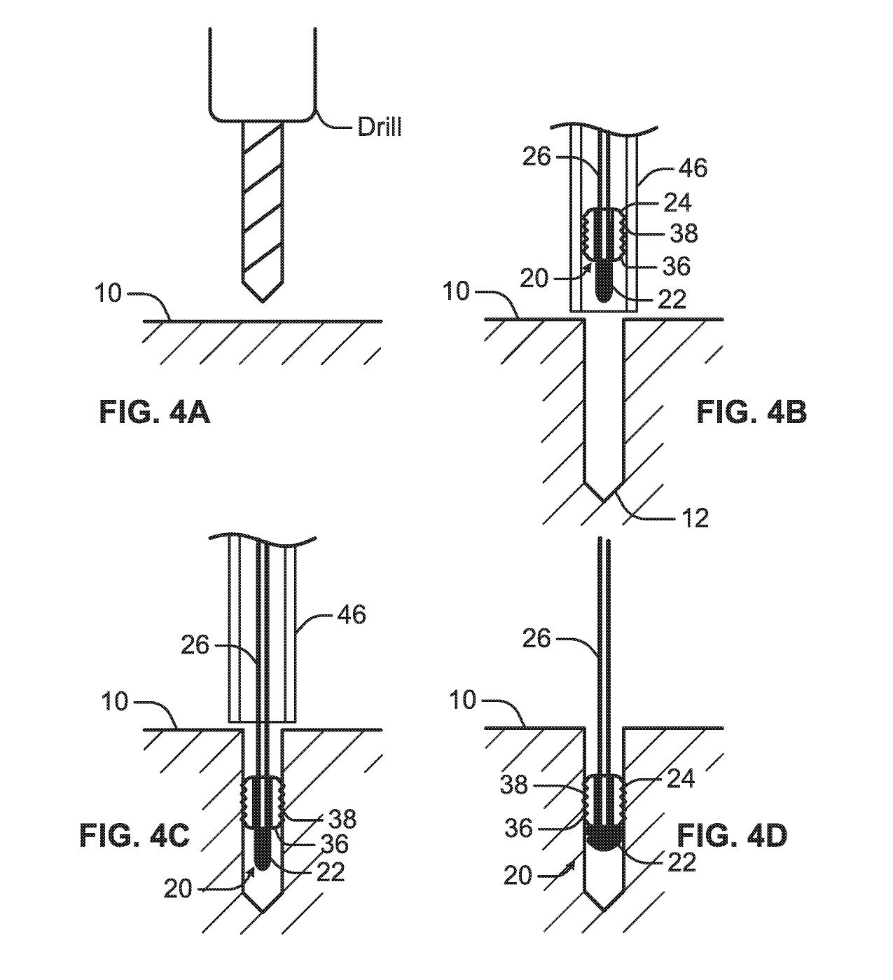

As depicted in FIGS. 3 and 4A-4D, in an example, a method of anchoring a suture 26 to a bone 10 can include an anchor site preparation step 50, an insertion step 52, an initial fixation step 54 and a final fixation step 56.

As depicted in FIG. 4A, the anchor site preparation step 50 can include drilling a bore hole 12 in the bone 10. In at least one example, a hole can be formed in the soft tissue to access the bore hole 12.

As depicted in FIG. 4B, the insertion step 52 can include delivering the suture anchor system 20 within a cannula 46 to the bore hole 12. The anchor system 20 can be advanced axially, nose portion 22 first and tail portion 24 second, into the bore hole 12. In at least one example, an inner cannula or other tool can extend through the cannula 46 to push the tail portion 24 and correspondingly the nose portion 22 into the bore hole 12.

As depicted in FIG. 4C, the initial fixation step 54 can include fixing the tail portion 24 to the inner surface 14 of the bore hole 12. In an example, the body 34 can be sized such that insertion of the body 34 into the bore hole 12 frictionally engages the outer surface 36 to the inner surface 14 of the bore hole 12 to fix the tail portion 24 within the bore hole 12. In at least one example, the fixation feature 38 positioned on the outer surface 36 can engage the bone defining the inner surface 14 of the bore hole 12 to fix the tail portion 24 within the bore hole 12. In this configuration, the nose portion 22 can be positioned on the interior side of the tail portion 24 such that the tail portion 24 retains the nose portion 22 within the bore hole 12.

As depicted in FIG. 4D, the final fixation step 56 can include tensioning the suture 26 through the internal channel 40 of the tail portion 24 to bunch up the flexible element 28 in a ball or bell-like configuration such that the flexible element 28 extends radially outward such that the flexible element 28 engages the inner surface 14 of the bore hole 12. The suture 26 can be tensioned by pulling the suture 26 away from the tail portion 24. In at least one example, an end of the suture 26 can be pulled away from the knot 42 to apply tension of the flexible element 28. The radial expansion of the flexible element 28 can fix the nose portion 22 within the bore hole 14. The tensioning of the suture 26 can also move the flexible element 28 axially within the bore hole 14 such that the flexible element 28 braces against the tail portion 24. The tail portion 24 can operate as a backstop for the flexible element 28 to prevent the flexible element 28 from being pulled from the bore hole 12 when the suture 26 is tensioned. The tail portion 24 can also facilitate further radial expansion of the flexible element 28 to improve engagement of the flexible element 28 to the inner surface 14 of the bore hole 12. The combined engagement of the nose portion 22 and the tail portion 24 can improve the retention of the anchor system 20 within the bore hole 14.

### VARIOUS NOTES & EXAMPLES

Example 1 is a suture anchor for anchoring a suture to a bore hole of a bone, comprising: a nose portion including a flexible element, the flexible element defining a longitudinal bore; and a tail portion including a body having an outer surface and defining an internal channel, wherein the body is configured to engage an inner surface of the bore hole to fix the tail portion within the bore hole; wherein a suture is insertable through the internal channel of the tail portion and through the longitudinal bore of the flexible element to operably couple the suture to the nose portion such that the suture can be tensioned through the internal channel of the tail portion to move the flexible element axially toward the tail portion such that the flexible element radially expands against the tail portion.

In Example 2, the subject matter of Example 1 optionally includes wherein the tail portion is sized such that the outer surface of the tail portion approximates the diameter of the bore; wherein the outer surface of the tail portion fictionally engages the tail portion to inner surface of the bore hole.

In Example 3, subject matter of any one or more of Examples 1-2 optionally include wherein the tail portion further comprises: a fixation feature extending radially outward from the outer surface of the body to engage the inner surface of the bore hole.

In Example 4, subject matter of Example 3 optionally includes wherein the fixation feature comprises a barbed ring oriented to permit insertion of the tail portion and engage the inner surface of the bore hole to prevent withdrawal of the tail portion.

In Example 5, subject matter of any one or more of Examples 3-4 optionally include wherein the fixation feature comprises an angled radial projection shaped to permit insertion of the tail portion and engage the inner surface of the bore hole to prevent withdrawal of the tail portion.

In Example 6, subject matter of any one or more of Examples 1-5 optionally include wherein the body of the tail portion further comprises at least one of a side opening and a recess to improve engagement of the outer surface of the body to the inner surface of the bore hole.

In Example 7, subject matter of any one or more of Examples 1-6 optionally include wherein the inner surface of the tail portion defines at least one guide channel for guiding tensioning of the suture within the internal channel.

In Example 8, subject matter of any one or more of Examples 1-7 optionally include wherein the nose portion includes at least two apertures through which the suture is received into the longitudinal bore defined by the flexible element.

In Example 9, subject matter of Example 8 optionally includes wherein the apertures are positioned inward from ends of the flexible element.

Example 10 is a suture anchor system, comprising: a suture anchor including: a nose portion including a flexible element, the flexible element defining a longitudinal bore, and a tail portion including a body having an outer surface and defining an internal channel, wherein the body is sized such that the outer surface can engage an inner surface of the bore hole to fix the tail portion within the bore hole; and a suture threaded through the internal channel of the tail portion through and into the longitudinal bore of the nose portion; wherein the suture is configured to be tensioned through the internal channel of the tail portion to move the flexible element axially toward the tail portion such that the flexible element radially expands against the inner surface of the bore hole.

In Example 11, subject matter of Example 10 optionally includes wherein the tail portion is sized such that the outer surface of the tail portion approximates the diameter of the bore; wherein the outer surface of the tail portion fictionally engages the tail portion to inner surface of the bore hole.

In Example 12, subject matter of any one or more of Examples 10-11 optionally include wherein the tail portion further comprises: a fixation feature extending radially outward from the outer surface of the body to engage the inner surface of the bore hole.

In Example 13, subject matter of Example 12 optionally includes wherein the fixation feature comprises a barbed ring oriented to permit insertion of the tail portion and engage the inner surface of the bore hole to prevent withdrawal of the tail portion.

In Example 14, subject matter of any one or more of Examples 12-13 optionally include wherein the fixation feature comprises an angled radial projection shaped to permit insertion of the tail portion and engage the inner surface of the bore hole to prevent withdrawal of the tail portion.

In Example 15, subject matter of any one or more of Examples 10-14 optionally include wherein the body of the tail portion further comprises at least one of a side opening and a recess to improve engagement of the outer surface of the body to the inner surface of the bore hole.

In Example 16, subject matter of any one or more of Examples 10-15 optionally include wherein the inner surface of the tail portion defines at least one guide channel for guiding tensioning of the suture within the internal channel.

In Example 17, subject matter of any one or more of Examples 10-16 optionally include wherein the nose portion includes at least two apertures through which the suture is received into the longitudinal bore defined by the flexible element.

In Example 18, subject matter of Example 17 optionally includes wherein the apertures are positioned inward from ends of the flexible element.

Example 19 is a method of anchoring a suture to a bone, comprising: threading a suture through an internal channel of a tail portion and looped through a longitudinal bore defined by a flexible element of a nose portion; inserting the nose portion into a bore hole formed in a bone; inserting the tail portion into the bore hole such that the tail portion engages an inner surface of the bore hole to fix the tail portion within the bore hole; tensioning the suture to draw the flexible element against the tail portion to radially expand the flexible element against the inner surface of the bore hole to fix the nose portion within the bore hole.

In Example 20, subject matter of Example 19 optionally includes wherein the tail portion includes a fixation feature extending radially outward from the outer surface of the body to engage the inner surface of the bore hole.

Example 21 is a method of anchoring a suture to a bone, comprising: threading a suture through an internal channel of a tail portion; looping the suture through a longitudinal bore defined by a flexible element of a nose portion; inserting the tail portion into the bore hole such that the tail portion engages an inner surface of the bore hole to fix the tail portion within the bore hole; inserting the nose portion through the internal channel of the tail portion into a bore hole formed in a bone; and tensioning the suture to draw the flexible element against the tail portion to radially expand the flexible element against the inner surface of the bore hole to fix the nose portion within the bore hole.

In Example 22, subject matter of Example 21. optionally includes wherein the tail portion includes a fixation feature extending radially outward from the outer surface of the body to engage the inner surface of the bore hole.

Each of these non-limiting examples can stand on its own, or can be combined in any permutation or combination with any one or more of the other examples.

The above detailed description includes references to the accompanying drawings, which form a part of the detailed description. The drawings show, by way of illustration, specific embodiments in which the present subject matter can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. If is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the present subject matter should be determined with reference to the appended claims, along with the full scope of equivalents to which such claims are entitled.

What is claimed is:

1. A suture anchor for anchoring a suture to a bore hole of a bone, comprising:
a nose portion including a flexible element, the flexible element defining a longitudinal bore; and
a tail portion including a body having an outer surface and defining an internal channel, wherein the body is configured to engage an inner surface of the bore hole to fix the tail portion within the bore hole;
wherein a suture is insertable through the internal channel of the tail portion and through the longitudinal bore of the flexible element to operably couple the suture to the nose portion such that the suture can be tensioned through the internal channel of the tail portion to move the flexible element axially toward the tail portion such that the flexible element radially expands against the tail portion.

2. The suture anchor of claim 1, wherein the tail portion is sized such that the outer surface of the tail portion approximates the diameter of the bore;
wherein the outer surface of the tail portion fictionally engages the tail portion to inner surface of the bore hole.

3. The suture anchor of claim 1, wherein the tail portion further comprises:
a fixation feature extending radially outward from the outer surface of the body to engage the inner surface of the bore hole.

4. The suture anchor of claim 3, wherein the fixation feature comprises a barbed ring oriented to permit insertion of the tail portion and engage the inner surface of the bore hole to prevent withdrawal of the tail portion.

5. The suture anchor of claim 3, wherein the fixation feature comprises an angled radial projection shaped to permit insertion of the tail portion and engage the inner surface of the bore hole to prevent withdrawal of the tail portion.

6. The suture anchor of claim 1, wherein the body of the tail portion further comprises at least one of a side opening and a recess to improve engagement of the outer surface of the body to the inner surface of the bore hole.

7. The suture anchor of claim 1, wherein the inner surface of the tail portion defines at least one guide channel for guiding tensioning of the suture within the internal channel.

8. The suture anchor of claim 1, wherein the nose portion includes at least two apertures through which the suture is received into the longitudinal bore defined by the flexible element.

9. The suture anchor of claim 8, wherein the apertures are positioned inward from ends of the flexible element.

10. A suture anchor system, comprising:
a suture anchor including:
a nose portion including a flexible element, the flexible element defining a longitudinal bore, and
a tail portion including a body having an outer surface and defining an internal channel, wherein the body is sized such that the outer surface can engage an inner surface of the bore hole to fix the tail portion within the bore hole; and
a suture configured to be threaded through the internal channel of the tail portion and into the longitudinal bore of the nose portion;
wherein the suture is configured to be tensioned through the internal channel of the tail portion to move the flexible element axially toward the tail portion such that the flexible element radially expands against the inner surface of the bore hole.

11. The suture anchor system of claim 10, wherein the tail portion is sized such that the outer surface of the tail portion approximates the diameter of the bore;
wherein the outer surface of the tail portion fictionally engages the tail portion to inner surface of the bore hole.

12. The suture anchor system of claim 10, wherein the tail portion further comprises:
a fixation feature extending radially outward from the outer surface of the body to engage the inner surface of the bore hole.

13. The suture anchor system of claim 12, wherein the fixation feature comprises a barbed ring oriented to permit insertion of the tail portion and engage the inner surface of the bore hole to prevent withdrawal of the tail portion.

14. The suture anchor system of claim 12, wherein the fixation feature comprises an angled radial projection shaped to permit insertion of the tail portion and engage the inner surface of the bore hole to prevent withdrawal of the tail portion.

15. The suture anchor system of claim 10, wherein the body of the tail portion further comprises at least one of a side opening and a recess to improve engagement of the outer surface of the body to the inner surface of the bore hole.

16. The suture anchor system of claim 10, wherein the inner surface of the tail portion defines at least one guide channel for guiding tensioning of the suture within the internal channel.

17. The suture anchor system of claim 10, wherein the nose portion includes at least two apertures through which the suture is received into the longitudinal bore defined by the flexible element.

18. The suture anchor system of claim 17, wherein the apertures are positioned inward from ends of the flexible element.

19. A method of anchoring a suture to a bone, comprising:
threading a suture through an internal channel of a tail portion;
looping the suture through a longitudinal bore defined by a flexible element of a nose portion;
inserting the nose portion into a bore hole formed in a bone;
inserting the tail portion into the bore hole such that the tail portion engages an inner surface of the bore hole to fix the tail portion within the bore hole; and
tensioning the suture to draw the flexible element against the tail portion to radially expand the flexible element against the inner surface of the bore hole to fix the nose portion within the bore hole.

20. The method of claim 19, wherein the tail portion includes a fixation feature extending radially outward from the outer surface of the body to engage the inner surface of the bore hole.

21. A method of anchoring a suture to a bone, comprising:
threading a suture through an internal channel of a tail portion;
looping the suture through a longitudinal bore defined by a flexible element of a nose portion;
inserting the tail portion into the bore hole such that the tail portion engages an inner surface of the bore hole to fix the tail portion within the bore hole;
inserting the nose portion through the internal channel of the tail portion into a bore hole formed in a bone; and
tensioning the suture to draw the flexible element against the tail portion to radially expand the flexible element against the inner surface of the bore hole to fix the nose portion within the bore hole.

22. The method of claim 21, wherein the tail portion includes a fixation feature extending radially outward from the outer surface of the body to engage the inner surface of the bore hole.
